(19) United States
(12) Patent Application Publication
(10) Pub. No.: US 2009/0069630 A1
(43) Pub. Date: Mar. 12, 2009

(54) CAPSULE TYPE MEDICAL DEVICE

(75) Inventor: Masatoshi Homan, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/200,460

(22) Filed: Aug. 28, 2008

(30) Foreign Application Priority Data
Aug. 30, 2007 (JP) 2007-224767
Aug. 30, 2007 (JP) 2007-224771

(51) Int. Cl. A61B 1/00

(52) U.S. Cl. 600/117

(57) ABSTRACT

A capsule type medical device, which is introduced into the inside of a living body in order to observe the inside of the living body, includes an image capturing unit for capturing an image of the inside of the living body, a wireless communicating unit for transmitting the image data of the image captured by the image capturing unit to the outside of the living body through a wireless communication, a first voltage regulator unit for regulating a voltage output from a power supply unit and for supplying the voltage to the image capturing unit, and a second voltage regulator unit for regulating the voltage output from the power supply unit and for supplying the voltage to the wireless communicating unit. An oscillating unit included in the wireless communicating unit has a differential configuration.

### CROSS REFERENCE TO RELATED APPLICATIONS

This application is based upon and claims the benefit of priority from the prior Japanese Patent Application No. 2007-224767 filed Aug. 30, 2007 and Japanese Patent Application No. 2007-224771 filed Aug. 30, 2007, the entire contents of which are incorporated herein by this reference.

### BACKGROUND OF THE INVENTION

1. Field of the Invention

The present invention relates to a capsule type medical device, which takes the shape of a capsule, is introduced into the inside of a living body in order to observe the inside of the living body, and wirelessly transmits image data captured inside the living body to a receiving device outside the living body, and more particularly, to a technique for enabling image data to be stably transmitted even while an image is being captured.

2. Description of the Related Art

Conventionally, various types of capsule type medical devices such as a capsule type endoscope, etc., which take the shape of a small capsule in order to observe the inside of a living body, especially, the inside of an organ of a digestive system of a patient, move sequentially inside the living body by being introduced from the mouth of the patient, and wirelessly transmit image data of the inside of the living body, which is captured during the moving, to a receiving device outside the living body are proposed.

Such capsule type medical devices mainly include an image capturing device such as a CCD camera, etc., which is intended to capture an image, and a transmitting device for wirelessly transmitting image data captured by the image capturing device to a receiving device outside the living body. In these capsule type medical devices, the image capturing device and the transmitting device are operated by using a button battery such as a 1.5-V silver oxide battery, etc. as a power supply.

### SUMMARY OF THE INVENTION

A capsule type medical device in one aspect of the present invention is a capsule type medical device, which is introduced into the inside of a living body in order to observe the inside of the living body. This capsule type medical device includes an image capturing unit for capturing an image of the inside of the living body, a wireless communicating unit for transmitting image data of the image captured by the image capturing unit to the outside of the living body through a wireless communication, a first voltage regulator unit for regulating a voltage output from a power supply unit and for supplying the voltage to the image capturing unit, and a second voltage regulator unit for regulating the voltage output from the power supply unit and for supplying the voltage to the wireless communicating unit, wherein an oscillating unit included in the wireless communicating unit has a differential configuration.

### BRIEF DESCRIPTION OF THE DRAWINGS

FIG. 1 is a block diagram showing a capsule type medical device according to a first embodiment of the present invention;

FIG. 2 is a block diagram showing a wireless communicating unit in the first embodiment according to the present invention;

FIG. 3 is a circuit diagram showing an oscillating unit in the first embodiment according to the present invention;

FIG. 4 is a block diagram showing a capsule type medical device according to a second embodiment of the present invention;

FIG. 5 is a block diagram showing a wireless communicating unit in the second embodiment according to the present invention; and

FIG. 6 is a circuit diagram showing an oscillating unit in the second embodiment according to the present invention.

### DESCRIPTION OF THE PREFERRED EMBODIMENTS

Embodiments according to the present invention are described below with reference to the drawings.

A first embodiment according to the present invention is described first.

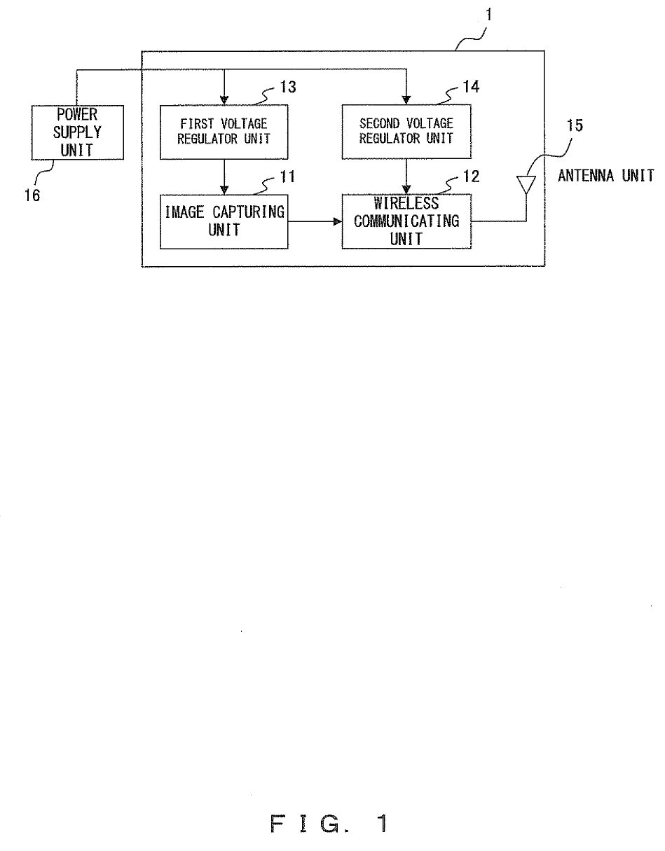

FIG. 1 is a block diagram showing a capsule type medical device according to the first embodiment of the present invention.

In FIG. 1, the capsule type medical device 1 is a capsule type endoscope, etc., which takes the shape of a small capsule in order to observe the inside of a living body, especially, the inside of an organ of a digestive system of a patient, sequentially moves inside the living body by being introduced from the mouth of the patient, and wirelessly transmits the image data of the inside of the living body, which is captured during the moving, to a receiving device outside the living body. The capsule type medical device 1 includes an image capturing unit 11 such as a CCD camera, etc., a wireless communicating unit 12, a first voltage regulator unit 13, a second voltage regulator unit 14, and an antenna unit 15.

The image capturing unit 11 captures an image of the inside of the living body while the capsule type medical device 1 is moving and staying inside the living body.

The wireless communicating unit 12 transmits the image data of the image captured by the image capturing unit 11 to the receiving device outside the living body through a wireless communication via the antenna unit 15.

The first voltage regulator unit 13 regulates a voltage output from a power supply unit 16 such as a button battery, etc., and supplies the voltage to the image capturing unit 11.

The second voltage regulator unit 14 regulates the voltage output from the power supply unit 16, and supplies the voltage to the wireless communicating unit 12.

Additionally, the voltage regulated by the second voltage regulator unit 14 is set to a voltage lower than the first voltage regulator unit 13 so that a voltage drop in the power supply unit 16 such as a button battery, etc. exerts little influence on the image capturing unit 11.

As described above, the voltage output from the same power supply unit 16 is regulated by the first voltage regulator unit 13 for the image capturing unit 11, and also regulated by the second voltage regulator unit 14 for the wireless communicating unit 12. Therefore, a stable voltage can be supplied to the wireless communicating unit 12 even if the operation mode of the image capturing unit 11 is switched while the wireless communicating unit 12 is operating.

The second voltage regulator unit 14 may be configured on the same IC as the wireless communicating unit 12.

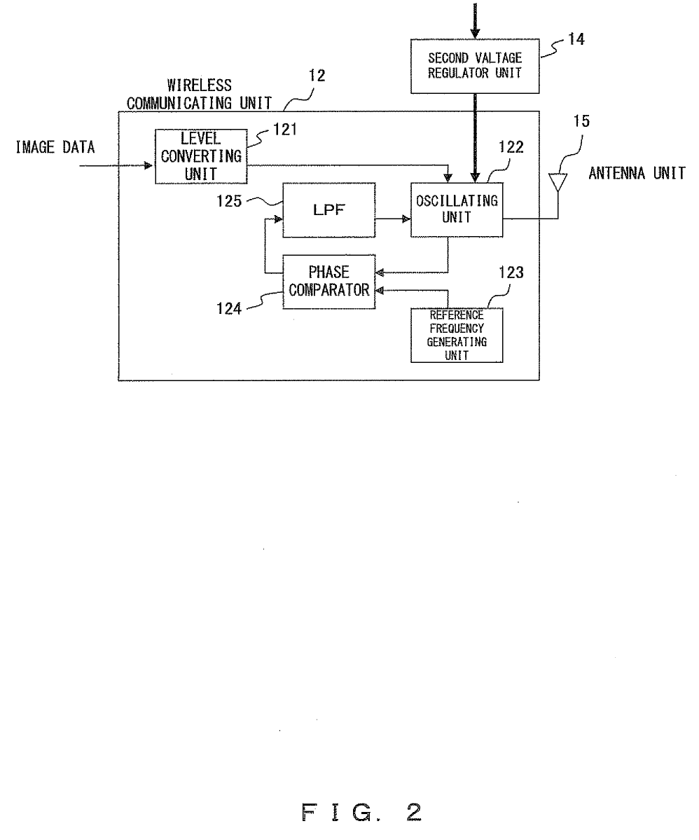

FIG. 2 is a block diagram showing the wireless communicating unit in the first embodiment according to the present invention.

In FIG. 2, the wireless communicating unit 12 includes a level converting unit 121, an oscillating unit 122, a reference frequency generating unit 123, a phase comparator 124, and a low-pass filter (LPF) 125.

The level converting unit 121 converts the voltage level of image data captured by the image capturing unit 11, and inputs the voltage level to the oscillating unit 122.

The oscillating unit 122 oscillates and outputs a signal at a frequency based on the voltage level of the image data after being converted, which is input from the level converting unit 121, and the voltage level of the signal output from the LPF 125 at the voltage regulated by the second voltage regulator unit 14. This oscillating unit 122 has a PLL (Phase Locked Loop) modulation circuit not shown, and also serves as an FSK (Frequency Shift Keying) modulator. Moreover, the frequency component of the input image data is set to a frequency higher than the cutoff frequency of the LPF 125.

The reference frequency generating unit 123 generates a reference frequency.

The phase comparator 124 makes a comparison between the phase of the signal output from the oscillating unit 122 and that of the reference frequency generated by the reference frequency generating unit 123, and outputs to the LPF 125 a signal according to a phase difference based on the result of the comparison.

The LPF 125 passes a low-frequency component of the input signal, and outputs the signal to the oscillating unit 122.

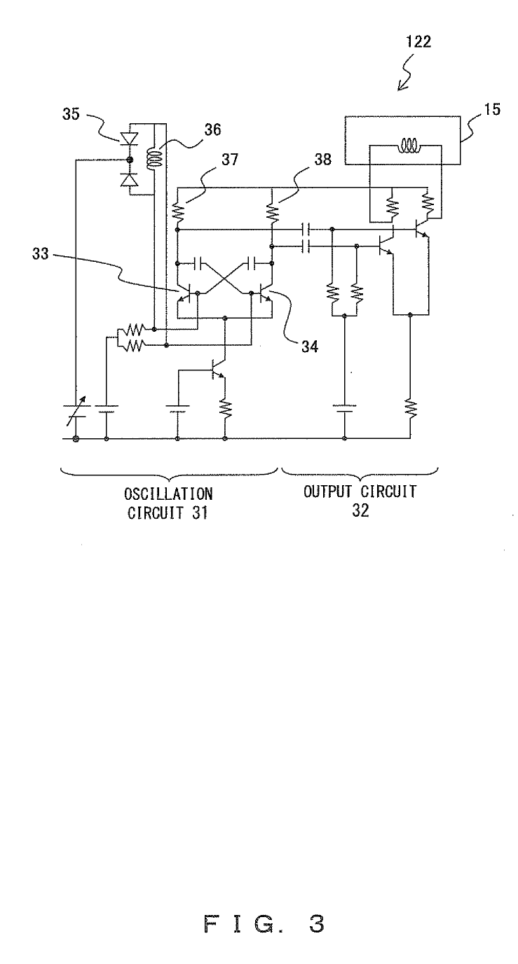

FIG. 3 is a circuit diagram showing the oscillating unit in the first embodiment according to the present invention.

In FIG. 3, the oscillating unit 122 is composed of an oscillation circuit 31 and an output circuit 32. A signal oscillated and output by the oscillation circuit 31 is output as radio waves via the output circuit 32 and the antenna unit 15. The antenna unit 15 is preferably a loop antenna or a dipole antenna, which is a balance type antenna, so that the signal can be efficiently output as radio waves from the oscillation circuit 31 having a differential configuration.

In the oscillation circuit 31, the base and the collector of one of transistors 33 and 34 are respectively cross-connected to the collector and the base of the other, and an output signal is output from the ground point of the two transistors 33 and 34. The cross-connections of the collectors and the bases of the two transistors 33 and 34 oscillate at the resonant frequency of an LC parallel resonant circuit. If an oscillation is made with a circuit having such a differential configuration, the influence of a ripple is reduced. Moreover, the oscillation circuit 31 having this differential configuration is an oscillation circuit of a base-resonant type.

Unlike a capsule type medical device according to a second embodiment to be described later, a varicap diode 35 and a coil 36 are connected on the side of the base in the oscillation circuit 31 included in the capsule type medical device 1. Moreover, not coils but resistors 37 and 38 are connected for a pull-up in the power supply.

As described above, the capsule type medical device 1 according to the first embodiment of the present invention can improve the resistance to a power supply ripple by implementing the oscillation circuit 31 as a differential configuration. Additionally, by providing the dedicated second voltage regulator unit 14 that is different from the first voltage regulator unit 13, which regulates the voltage supplied to the image capturing unit 11, on a power supply line for supplying the voltage to the oscillation circuit 31 included in the wireless communicating unit 12, a power supply ripple that occurs in the image capturing unit 11 can be removed as much as possible.

Furthermore, by combining these two configurations, namely, according to the two characteristics that the capsule type medical device 1 is provided with the first voltage regulator unit 13 for stabilizing the voltage supplied to the image capturing unit 11, and the second voltage regulator unit 14 for stabilizing the voltage supplied to the wireless communicating unit 12, and that the oscillating unit 122 is implemented as a differential configuration, the degree of stability of the frequency of the wireless communicating unit 12 can be improved, and also the quality of the wireless communication can be improved. Accordingly, the capsule type medical device 1 according to the present invention can stably transmit image data even if the operation mode of the image capturing unit 11 is switched while the wireless communicating unit 12 is operating.

The second embodiment according to the present invention is described next.

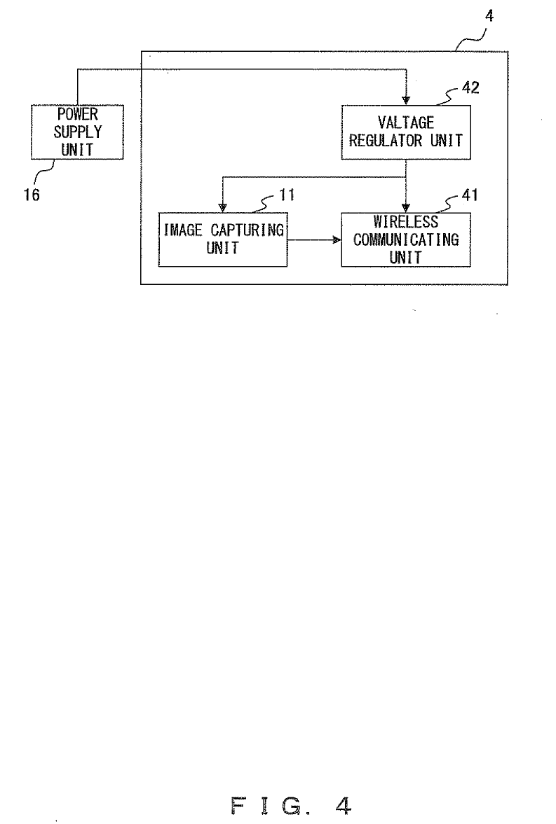

FIG. 4 is a block diagram showing a capsule type medical device according to the second embodiment of the present invention.

In FIG. 4, similar to the capsule type medical device 1 according to the first embodiment described with reference to FIG. 1, the capsule type medical device 4 is a capsule type endoscope, etc., which takes the shape of a small capsule in order to observe the inside of a living body, especially, the inside of an organ of a digestive system of a patient, sequentially moves inside the living body by being introduced from the mouth of the patient, and wirelessly transmits the image data of the inside of the living body, which is captured during the moving, to a receiving device outside the living body. The capsule type medical device 4 includes the image capturing unit 11 such as a CCD camera, etc., a wireless communicating unit 41, and a voltage regulator unit 42.

The image capturing unit 11 captures an image of the inside of the living body while the capsule type medical device 4 is moving and staying inside the living body.

The wireless communicating unit 41 transmits the image data of the image captured by the image capturing unit 11 to the receiving device outside the living body through a wireless communication via an incorporated antenna unit 15 of a differential type.

The voltage regulator unit 42 regulates a voltage output from the power supply unit 16 such as a button battery, etc., and supplies the voltage to both the image capturing unit 11 and the wireless communicating unit 41.

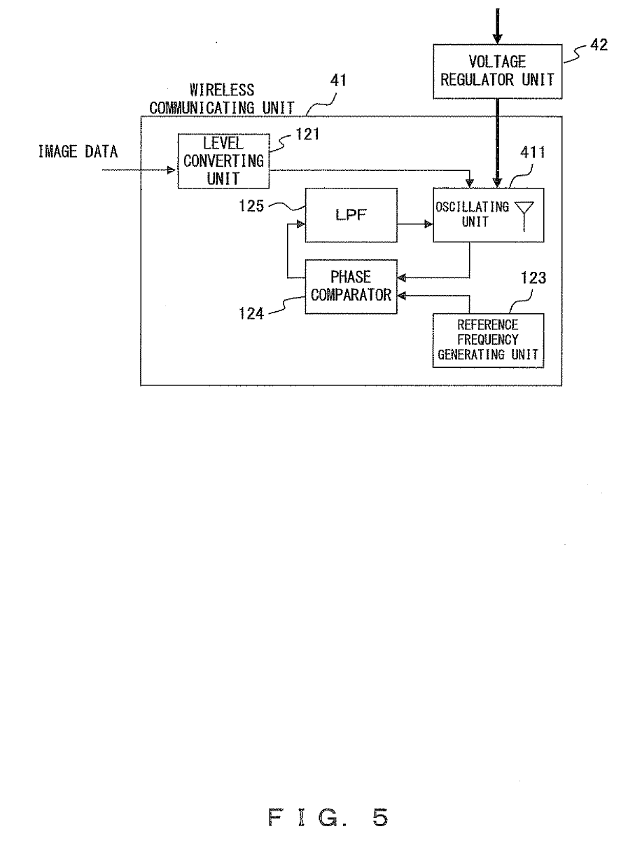

FIG. 5 is a block diagram showing the wireless communicating unit in the second embodiment according to the present invention.

In FIG. 5, the wireless communicating unit 41 includes the level converting unit 121, an oscillating unit 411, the reference frequency generating unit 123, the phase comparator 124, and the low-pass filter (LPF) 125.

The level converting unit 121 converts the voltage level of image data captured by the image capturing unit 11, and inputs the voltage level to the oscillating unit 411.

The oscillating unit 411 oscillates and outputs a signal based on the voltage level of the image data after being converted, which is input from the level converting unit 121, and the voltage level of the signal output from the LPF 125 at the voltage regulated by the voltage regulator unit 42. This oscillating unit 411 incorporates an antenna unit of a balance type, includes a PLL modulation circuit not shown, and also serves as an FSK modulator similar to the oscillating unit 122 in the first embodiment described with reference to FIG. 2. Moreover, the frequency component of image data is set to a frequency higher than the cutoff frequency of the LPF 125.

The reference frequency generating unit 123 generates a reference frequency.

The phase comparator 124 makes a comparison between the phase of the signal output from the oscillating unit 411 and that of the reference frequency generated by the reference frequency generating unit 123, and outputs to the LPF 125 a signal according to a phase difference based on the result of the comparison.

The LPF 125 passes a low-frequency component of the input signal, and outputs the signal to the oscillating unit 411.

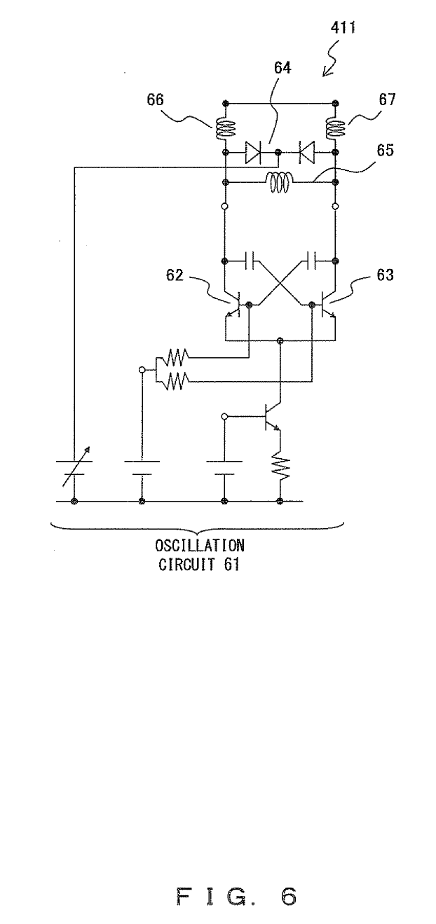

FIG. 6 is a circuit diagram showing the oscillating unit in the second embodiment according to the present invention.

In FIG. 6, the oscillating unit 411 is composed of an oscillation circuit 61, and an oscillation inductor arranged between collector terminals is made to also serve as an antenna unit 65 for generating an electromagnetic field. A signal oscillated and output by the oscillation circuit 61 is output as radio waves via the antenna unit 65. The shape of the antenna unit 65 is preferably a loop or a spiral having an outer diameter of 3 mm or longer, which can form an inductor.

In the oscillation circuit 61, the base and the collector of one of transistors 62 and 63 are respectively cross-connected to the collector and the base of the other, and an output signal is output from the ground point of the two transistors 62 and 63. The cross-connections of the collectors and the bases of the two transistors 62 and 63 oscillate at the resonant frequency of an LC parallel resonant circuit. If an oscillation is made with a circuit having such a differential configuration, the influence of a ripple is reduced. Moreover, the oscillation circuit 61 having this differential configuration is an oscillation circuit of a collector-resonant type.

Unlike the above described capsule type medical device 1 according to the first embodiment, a varicap diode 64 and a coil as the antenna unit 65 are connected on the side of the collector in order to make the coil also serve as the externally extending antenna unit 65 in the oscillation circuit 61 included in the capsule type medical device 4. Moreover, not resistors but coils 66 and 67 are connected for a pull-up in the power supply.

As described above, the capsule type medical device 4 according to the second embodiment of the present invention can improve the resistance to a power supply ripple by implementing the oscillation circuit 61 as a differential configuration.

Additionally, by implementing the oscillation circuit 61 and the antenna unit 65 as a collector-resonant type, the coil for resonance can be made to also serve as that for the antenna. This eliminates the need for the output circuit, leading to reductions in the power consumption and the size of the capsule type medical device 4.

As described above, according to the present invention, image data can be stably transmitted even if the operation mode of the image capturing unit is switched while the wireless communicating unit is operating, and power consumed by the operations of the transmitting device can be reduced.

The embodiments according to the present invention have been described up to this point. However, capsule type medical devices according to the present invention are not limited to the above described embodiments as far as their functions are carried out, and can take various configurations or shapes within a scope that does not depart from the gist of the present invention.

For example, the capsule type medical device 4 according to the second embodiment may be configured so that the voltage regulator unit 42 regulates only the voltage supplied to the wireless communicating unit 41, and a voltage regulator like the first voltage regulator 13 in the first embodiment, which regulates the voltage supplied to the image capturing unit 11, is provided separately from the voltage regulator 42.

What is claimed is:

1. A capsule type medical device introduced into an inside of a living body in order to observe the inside of the living body, comprising:
an image capturing unit for capturing an image of the inside of the living body;
a wireless communicating unit for transmitting image data of the image captured by the image capturing unit to an outside of the living body through a wireless communication;
a first voltage regulator unit for regulating a voltage output from a power supply unit, and for supplying the voltage to the image capturing unit; and
a second voltage regulator unit for regulating the voltage output from the power supply unit, and for supplying the voltage to the wireless communicating unit, wherein
an oscillating unit comprised by the wireless communicating unit has a differential configuration.

2. The capsule type medical device according to claim 1, wherein
a value of the voltage supplied by the second voltage regulator unit is smaller than a value of the voltage supplied by the first voltage regulator unit.

3. The capsule type medical device according to claim 2, wherein
the oscillating unit is of a base-resonant scheme.

4. The capsule type medical device according to claim 1, wherein
the oscillating unit has a PLL (Phase Locked Loop), and also serves an FSK (Frequency Shift Keying) modulation function.

5. The capsule type medical device according to claim 2, wherein
the oscillating unit is of a collector-resonant scheme.

6. The capsule type medical device according to claim 5, wherein
an oscillation inductor arranged between collector terminals of the oscillating unit is made to also serve as an antenna for generating an electromagnetic field, which is comprised by the wireless communicating unit.

7. A capsule type medical device introduced into an inside of a living body in order to observe the inside of the living body, comprising:
image capturing means for capturing an image of the inside of the living body;
wireless communicating means for transmitting image data of the image captured by the image capturing means to an outside of the living body through a wireless communication;
first voltage regulator means for regulating a voltage output from power supply means, and for supplying the voltage to the image capturing means; and
second voltage regulator means for regulating the voltage output from the power supply means, and for supplying the voltage to the wireless communicating means, wherein
an oscillating unit comprised by the wireless communicating means has a differential configuration.
